(19) United States
(12) Patent Application Publication
(10) Pub. No.: US 2024/0241136 A1
(43) Pub. Date: Jul. 18, 2024

(54) DIAGNOSTIC ASSAYS FOR MOVEMENT DISORDERS

(71) Applicant: UNIVERSITY HEALTH NETWORK, Toronto (CA)

(72) Inventors: Ivan MARTINEZ-VALBUENA, Toronto (CA); Gabor G. KOVACS, Toronto (CA); Anthony E. LANG, Toronto (CA)

(21) Appl. No.: 18/537,455

(22) Filed: Dec. 12, 2023

Related U.S. Application Data
(60) Provisional application No. 63/438,423, filed on Jan. 11, 2023.

(30) Foreign Application Priority Data
Jan. 11, 2023 (CA) 3186241

(51) Int. Cl. G01N 33/68

(52) U.S. Cl. CPC G01N 33/6896; G01N 2333/4728; G01N 2800/2835

(57) ABSTRACT

In an aspect, there is provided a method for differentiating movement disorders in a subject, comprising performing a seeding amplification assay for alpha-synuclein in a subject sample utilizing a specific buffer conditions, measuring the alpha-synuclein aggregation of the sample in the assay based on at least one of the following kinetic parameters: lag time, growth phase, T50, ThT max and AUC; and differentiating between Parkinson's Disease (PD) and Multiple System Atrophy (MSA) based on said sample kinetic parameters compared to control kinetic parameters, wherein control kinetic parameters for MSA show at least a two-fold difference from the control kinetic parameters for PD.

### CROSS REFERENCE TO RELATED APPLICATIONS

The application claims priority to U.S. Provisional Patent Application No. 63/438,423 filed on Jan. 11, 2023, and Canadian Patent Application No. 3,186,241 filed on Jan. 11, 2023, both of which are hereby incorporated by reference in their entirety.

### FIELD OF THE INVENTION

The invention relates to the diagnosis and treatment of movement disorders, such as Parkinson's Disease (PD), Multiple System Atrophy (MSA), and Progressive Supranuclear Palsy (PSP).

### BACKGROUND OF THE INVENTION

A rapid and non-invasive method to provide an early and accurate diagnosis and differentiation of neurodegenerative parkinsonisms remains a critical unmet need. The detection of disease-associated alpha-synuclein (aSyn) using Real-Time Quaking-induced Conversion (RT-QuIC) has shown extremely promising results in Parkinson's disease (PD) [37].

Multiple system atrophy (MSA) is a progressive neurodegenerative disorder with a clinical presentation of various combinations of parkinsonism, cerebellar and autonomic dysfunction [1]. The term MSA was coined in 1969 to pool previously described neurological entities [2], however, the major common finding of argyrophilic oligodendrocytic cytoplasmic inclusions (GCIs), called Papp-Lantos bodies contributed significantly to the nosological definition of the disease [3]. Presence of the 140 aa protein, α-synuclein as a major component of these inclusions linked MSA with Lewy body disorders such as Parkinson's disease (PD) and dementia with Lewy Bodies [4]. Collectively, these disorders are now termed synucleinopathies. Lewy body disorders are classically distinguished from MSA by distinct cellular pathology. Although both conditions accumulate α-synuclein in a variety of cell types, MSA is characterized by oligodendrocytic inclusions, while neuronal α-synuclein pathology predominates in Lewy body disorders. In addition, recent studies have uncovered the presence of α-synuclein “polymorphs”, suggestive of different strains as occurs in prion diseases, that might be the basis for the phenotypic diversity found in these conditions. The presence of different strains is hypothesized to dictate the cell-to-cell spreading of pathology and the cellular impact of the pathological α-synuclein in every individual [5,6]. Consistent with this notion, many experimental findings have indicated that α-synuclein forming GCIs has greater seeding activity compared to Lewy body (LB) associated α-synuclein [7,8]. Furthermore, recent discoveries using cryo-electron microscopy showed structural differences between the aggregates found in MSA and dementia with Lewy Bodies brains [9,10].

In recent years, the use of seeded amplification assays has emerged as a reliable method of detecting minute amounts of misfolded disease-associated proteins or seeds such as prion protein, tau or α-synuclein [11]. These assays, also known as Real-Time Quaking-induced Conversion (RT-QuIC) and protein misfolding cyclic amplification (PMCA), exploit the property of self-propagation to amplify and then sensitively detect minute amounts of these protein seeds [12,13]. Real-time detection of thioflavin T (ThT) fluorescence at multiple timepoints during the assay permits the measurement of kinetic differences between the seeding properties of different samples, providing a highly specific characterization of whether a disease-associated protein is present or not. However, despite the reliability of these assays to consistently detect α-synuclein seeds in Lewy body disorders, using a variety of biological samples [14-17], attempts to detect α-synuclein in MSA have proven challenging. Van Rumund et al. found that α-synuclein RT-QuIC was positive in only 6/17 cerebrospinal fluid (CSF) samples in MSA [16], while Rossi et al. detected an even lower number of RT-QuIC positive CSF samples in MSA (2/29) [17]. In contrast, using PMCA over a period of 350 hours, Shahawanaz et al. were able to detect α-synuclein seeding activity in 65/75 MSA CSF samples, but noted that in spite of aggregating faster, MSA CSF and brain samples reached a lower fluorescence plateau than PD CSF and brain samples [18].

Recently, several studies have also demonstrated that using RT-QuIC, aSyn seeding activity can be detected in skin biopsy homogenates from PD patients and distinguish them from controls with high specificity and sensitivity [38-41]. However, one of the major challenges in achieving early differentiation of neurodegenerative parkinsonisms has been the inconsistent detection of disease-associated aSyn in multiple system atrophy (MSA) patients' skin using RT-QuIC.

### SUMMARY OF THE INVENTION

In an aspect, there is provided a method for differentiating movement disorders in a subject, comprising:
performing a seeding amplification assay for alpha-synuclein in a subject sample utilizing one of the following buffer conditions:
(i) Trisodium citrate, pH 7-9;
(ii) NaF, pH 7-9;
(iii) NaCLO4, PH 3-5;
(iv) GndCl, pH 3-5; and
(v) MgCl2, pH 3-5;
measuring the alpha-synuclein aggregation of the sample in the assay based on at least one of the following kinetic parameters: lag time, growth phase, T50, ThT max and AUC;
differentiating between Parkinson's Disease (PD) and Multiple System Atrophy (MSA) based on said sample kinetic parameters compared to control kinetic parameters, wherein control kinetic parameters for MSA show at least a two-fold difference from the control kinetic parameters for PD.

In an aspect, there is provided a kit for performing a seeding amplification assay for alpha-synuclein, wherein the buffer included in said kit is as defined herein.

### BRIEF DESCRIPTION OF FIGURES

These and other features of the preferred embodiments of the invention will become more apparent in the following detailed description in which reference is made to the appended drawings wherein:

FIG. 1. Schematic of the RT-QuIC buffer discovery phase to evaluate α-synuclein seeding in MSA. 1.1) Samples containing α-synuclein seeds were prepared from substantia nigra of PD or cerebellum of MSA patients. 1.2) Samples were incubated with 168 reaction buffers and subjected to cycles of shaking and resting at 37° C. 1.3) ThT output was measured at multiple timepoints over 48 hours. 1.4) Results were analyzed and heatmaps were generated to determine the optimal conditions to discriminate MSA-derived samples from PD-derived samples. Designed with Biorender.com

FIG. 2. Conformational discrimination of α-synuclein strains in PD and MSA brain. a) Representative immunohistochemistry image for aggregated α-synuclein in the cerebellum white matter from a MSA patient showing GCIs. b) Representative immunohistochemistry image for aggregated α-synuclein in the SN from a PD patient showing Lewy bodies and Lewy neurites. c) Representative total α-synuclein (Syn-1 clone) immunoblot showing the thermolysin digestion (TL) of brain extracts from the cerebellum and the SN of a MSA and PD patient, respectively. d) CSA for α-synuclein aggregates in brain extracts from patients with MSA or PD. Representative α-synuclein immunoblots and the resultant denaturation curves (e) are shown. The curves depict mean residual α-synuclein values following treatment with the indicated concentrations of GdnCl. Higher GdnCl₅₀ values were obtained for α-synuclein aggregates in the patient with PD than for the patient with MSA.

FIG. 3. Changes in RT-QuIC physicochemical factors promote the detection of α-synuclein seeding in MSA. Fold separation of lag time, growth phase, T50, ThT max and area under the curve (AUC) between reactions seeded with MSA- or PD-derived α-synuclein aggregates and amplified under 4 different pH using 7 salts at 6 concentrations. Larger numbers (blue) represent the conditions that showed a more favorable environment to detect MSA α-synuclein aggregation. When the fold separation is 1 (deep red), no discrimination between MSA and PD aggregation was found. Lower numbers (<1, lighter red/purple) represent the conditions more favorable to detect PD aggregation over MSA. *Buffers highlighted in boxes represent the optimal conditions to detect α-synuclein seeding in MSA and discriminate from PD.

FIG. 4. Interaction of ThT dye with α-synuclein aggregates derived from patients with PD or MSA is dependent on the RT-QuIC reaction buffer used. a) Representative aggregation curves (median case) of α-synuclein in the presence of brain homogenates from patients with PD (n=15), patients with MSA (n=15), patients with supranuclear progressive palsy (n=5) and controls (n=5) amplified with the 50 mM Gly pH4 NaClO₄ buffer. Data are mean±s.e.m. of representative subjects measured in quadruplicate. Area under the curve values (b) maximum ThT fluorescence values (c) and T50 (d) for PD (n=15), MSA (n=15), supranuclear progressive palsy (n=5) and controls (n=5). Each dot represents an individual biological sample measured in quadruplicate. e) At the ultrastructural level, RT-QuIC-derived MSA-derived fibrils are thicker and more twisted than the PD-derived fibrils, as determined by electron microscopy. f) Aggregation profiles of α-synuclein in the presence of brain homogenates from patients with PD (n=15), patients with MSA (n=15), patients with supranuclear progressive palsy (n=5) and controls (n=5) amplified with the 40 mM PB pH8 Na₃Citrate buffer. Data are mean±s.e.m. of representative subjects measured in quadruplicate. Area under the curve values (g) maximum ThT fluorescence values (h) and T50 (i) for PD (n=15), MSA (n=15), supranuclear progressive palsy (n=5) and controls (n=5). Each dot represents an individual biological sample measured in quadruplicate. The grey dots in (h) indicate the MSA patients that were selected for the brain regional analysis according to their divergent seeding behavior. j) At the ultrastructural level, RT-QuIC-derived MSAfibrils are thicker and twisted than the PD-derived fibrils, as determined by electron microscopy. *P<0.05, **P<0.01, ***P<0.001, ****P<0.0001 by one-way analysis of variance (ANOVA) followed by Tukey's multiple comparison test (b, c, d, g, h, i). Scale bar (e, j): 25 nm.

FIG. 5. RT-QuIC reaction products seeded from MSA α-synuclein fibrils are conformationally distinguishable from RT-QuIC reaction products seeded from PD-derived fibrils. a) CSA for the RT-QuIC-derived MSA and PD fibrils. Representative α-synuclein immunoblots and the resultant denaturation curves (b) are shown. MSA-derived fibrils are less stable than PD-derived fibrils in the epitope (amino acids 15-123) used to probe the structure. c) Immunoblots of PBS-soluble α-synuclein species in brain homogenates from PD and MSA patients and their RT-QuIC-derived fibrils with digestion with thermolysin (TL).TL-resistant α-syn species were present in MSA and PD brain extracts, but TL-resistant α-syn species were only detectable in the MSA RT-QuIC-derived fibrils.

FIG. 6. Extensive heterogeneity in α-synuclein seeding activity is observed in different MSA patients and between different brain regions. Heat mapping of α-synuclein seeding (a) assessed by RT-QuIC and of aggregated α-synuclein (b) evaluated by immunohistochemistry using the conformational α-synuclein 5G4 antibody. The α-synuclein seeding ranges from white (none) through yellow (low) and orange (medium) to red (high). The semiquantitative score of the severity of α-synuclein pathology ranges from white (none) through yellow (mild) and orange (moderate) to red (severe). Grey colored cortical regions indicate that the region was not evaluated.

FIG. 7. Workflow for the identification of individuals with MSA and PD, with high sensitivity and specificity, from amongst a mixed population of individuals representative of those typically referred for assessment for a suspected parkinsonian disorder. *Sensitivity and specificity values were calculated assuming an exploratory NfL cut-off value of 30 pg/ml, that require validation in independent cohorts. Figure created with Biorender.com.

FIG. 8. Skin biopsy aSyn RT-QuIC and serum NfL levels. Serum NfL levels (pg/mL) are shown for each individual participant. The colour of the circles indicate the detection of aSyn seeding activity by RT-QuIC (green for positive and red for negative) in skin biopsy samples from individual participants (p) from each experimental group; a) healthy controls (none of the 3 positive cases gave a history of symptoms suggestive of prodromal Parkinson's disease (PD)), b) PD, c) multiple system atrophy (MSA) and d) progressive supranuclear palsy (PSP). e) Representative RT-QuIC aggregation curves of aSyn in the presence of skin homogenates from patients with PD, MSA, or PSP and controls. Data are mean±s.e.m. of representative subjects measured in quadruplicate. F) Serum NfL levels in patients with MSA and PSP were significantly enhanced in patients with PSP and MSA compared to controls and PD subjects.

### DETAILED DESCRIPTION

In the following description, numerous specific details are set forth to provide a thorough understanding of the invention. However, it is understood that the invention may be practiced without these specific details.

Multiple System Atrophy (MSA) is a neurodegenerative condition characterized by variable combinations of parkinsonism, autonomic failure, cerebellar ataxia and pyramidal features. Although the distribution of synucleinopathy correlates with the predominant clinical features, the burden of pathology does not fully explain observed differences in clinical presentation and rate of disease progression. We hypothesized that clinical heterogeneity in MSA is the consequence of variability in the seeding activity of α-synuclein both between different patients and between different brain regions.

The reliable detection of α-synuclein seeding activity derived from MSA using cell-free amplification assays has remained challenging.

These divergent findings are likely the result of different conditions in the reaction buffers used in these studies. Thus, in the present study, we sought to identify the optimal assay conditions that favor MSA seeding activity, by systematically modulating the ionic and cationic composition and the pH of the α-synuclein RT-QuIC reaction buffer. The effect of ionic composition and pH in α-synuclein aggregation has been widely studied [19], but not in the context of the systematic comparison of a spectrum of reaction buffers seeded with brain homogenates from MSA and PD.

Therefore, we conducted a systematic evaluation of 168 different reaction buffers, using an array of pH and salts, seeded with fully characterized brain homogenates from one MSA and one PD patient. We then validated the two conditions that conferred the optimal ability to discriminate between PD and MSA-derived samples in a larger cohort of 40 neuropathologically confirmed cases, including 15 MSA. Finally, in a subset of brains, we conducted the first multi-region analysis of seeding behaviour in MSA.

Using our novel buffer conditions, we show that the physicochemical factors that govern the in vitro amplification of α-synuclein can be tailored to generate strain-specific reaction buffers that can be used to reliably study the seeding capacity from MSA derived α-synuclein. Using this novel approach, we were able to sub-categorize the 15 MSA brains into 3 groups: high, intermediate and low seeders. To further demonstrate heterogeneity in α-synuclein seeding in MSA, we conducted a comprehensive multi-regional evaluation of α-synuclein seeding in 13 different regions from 2 high seeders, 2 intermediate seeders and 2 low seeders.

We have identified unexpected differences in seed-competent α-synuclein across a cohort of neuropathologically comparable MSA brains. Furthermore, our work has revealed a substantial heterogeneity in seeding activity, driven by the PBS soluble α-synuclein, between different brain regions of a given individual that goes beyond immunohistochemical observations. Our observations pave the way for the future subclassification of MSA, that exceeds conventional clinical and neuropathological phenotyping and considers the structural and biochemical heterogeneity of α-synuclein present. Finally, our methods provide an experimental framework for the development of vitally needed, rapid and sensitive diagnostic assays for MSA.

In instances where seeding activity is limited, such as in the case of skin biopsies, we also supplement our novel assay with circulating neurofilament light chain (NfL) as a potential early marker to distinguish patients with PD from those with atypical parkinsonian disorders [42].

In an aspect, there is provided a method for differentiating movement disorders in a subject, comprising:
performing a seeding amplification assay for alpha-synuclein in a subject sample utilizing one of the following buffer conditions:
(i) Trisodium citrate, pH 7-9;
(ii) NaF, pH 7-9;
(iii) NaCLO₄, PH 3-5;
(iv) GndCl, pH 3-5; and
(v) MgCl₂, pH 3-5;
measuring the alpha-synuclein aggregation of the sample in the assay based on at least one of the following kinetic parameters: lag time, growth phase, T50, ThT max and AUC;
differentiating between Parkinson's Disease (PD) and Multiple System Atrophy (MSA) based on said sample kinetic parameters compared to control kinetic parameters, wherein control kinetic parameters for MSA show at least a two-fold difference from the control kinetic parameters for PD.

Seed amplification assays (SAAs) are biophysical tools that take advantage on the peculiar properties of prion proteins by amplifying small amounts of aggregates in biological fluids at the expense of recombinant monomeric protein added in solution. Examples of SAAs include, without limitation, Real-Time Quaking-Induced Conversion and Protein Misfolding Cyclic Amplification.

As used herein, the term “control” refers to a specific value or dataset that can be used as a comparator to a measured value. Without limitation, this includes control values for kinetic parameters measured in seeding amplification assays or serum levels of neurofilament light chain (NfL) in subjects or subject datasets with known movement disorders.

The term “sample” as used herein refers to any fluid, cell or tissue sample from a subject that can be assayed for the proteins or other biomarkers measured by the present methods.

In some embodiments, the buffer condition is one of:
(i) Trisodium citrate, pH8;
(ii) NaF, about pH8;
(iii) NaCLO₄, about pH4;
(iv) GndCl, about pH4; and
(v) MgCl₂, about pH4;

In some embodiments, the buffer condition is one of:
(i) Trisodium citrate, about pH8, PB;
(ii) NaF, about pH8, PB;
(iii) NaCLO₄, about pH4, Glycine;
(iv) GndCl, about pH4, Glycine; and
(v) MgC₁₂, about pH4, Glycine;

In some embodiments, the buffer condition is one of:
(i) Trisodium citrate, about pH8, about 40 mM PB;
(ii) NaF, about pH8, about 40 mM PB;
(iii) NaCLO₄, about pH4, about 50 mM Glycine;
(iv) GndCl, about pH4, about 50 mM Glycine; and
(v) MgCl₂, about pH4, about 50 mM Glycine;

In some embodiments, the buffer condition is (i). Preferably, the concentration of Trisodium citrate is 50 nm to 500 nm. Further preferably, the concentration of Trisodium citrate is about 350 nm.

In some embodiments, the buffer condition is (ii). Preferably, the concentration of NaCLO₄ is about 200 nm to 300 nm. Further preferably, the concentration of NaCLO₄ is about 250 nm.

In some embodiments, the method further comprises the step of digesting the seeding amplification assay end products with thermolysin and wherein a MSA sample shows a 15 kDa product post-digestion and a PD sample does not show a distinct 15 kDa product post-digestion.

In some embodiments, the method further comprises the step of digesting the seeding amplification assay end products with GndCl and comparing to control end product stability; wherein a MSA sample exhibits less stability to GndCl digestion than a PD sample.

In some embodiments, the sample from the subject has demonstrated alpha-synuclein seeding activity and the method further comprises:
measuring serum neurofilament light chain (NfL) concentration in the subject; and
further differentiating between PD and MSA based on said NfL concentration compared to control NfL concentrations, wherein control NfL concentrations for MSA are significantly increased compared to control NfL concentrations for PD.

In some embodiments, the method is for additionally differentiating progressive supranuclear palsy (PSP) from MSA and PD, the method further comprising:
measuring serum neurofilament light chain (NfL) concentration in the subject; and
differentiating progressive supranuclear palsy (PSP) from MSA and PD, wherein the sample from the subject demonstrated no/low alpha-synuclein seeding activity and said wherein a NfL measured concentration is compared to control NfL concentrations, wherein control NfL concentrations for PSP are significantly increased compared to control NfL concentrations for PD.

In some embodiments, the seeding amplification assay is Real-Time Quaking-Induced Conversion.

In some embodiments, the seeding amplification assay is Protein Misfolding Cyclic Amplification.

In some embodiments, the subject sample is brain, cerebrospinal fluid, skin, blood, saliva, urine, sebum, nasal secretions or nasal mucosal membrane, and extracellular vesicles (EVs) isolated from any of the foregoing.

In some embodiments, the subject sample is brain. In specific embodiments, the brain tissue is hippocampus-derived or amygdala-derived.

In some embodiments, the subject sample is skin.

In some embodiments, if the subject has been determined to have Parkinson's Disease, the method further comprises treating the subject for Parkinson's Disease.

In some embodiments, if the subject has been determined to have MSA, the method further comprises treating the subject for MSA.

In some embodiments, if the subject has been determined to have PSP, the method further comprises treating the subject for PSP.

In an aspect, there is provided a kit for performing a seeding amplification assay for alpha-synuclein, wherein the buffer included in said kit is as defined in above.

In some embodiments, the kit further comprises reagents to measure serum neurofilament light chain (NfL) concentration in a subject.

The advantages of the present invention are further illustrated by the following examples. The examples and their particular details set forth herein are presented for illustration only and should not be construed as a limitation on the claims of the present invention.

### Example 1

### Methods and Materials

### Human Tissue Samples

15 subjects with MSA, 15 subjects with PD, 5 controls and 5 subjects with supranuclear progressive palsy were selected from the University Health Network-Neurodegenerative Brain Collection (UHN-NBC, Toronto, Canada) based on a definite neuropathological diagnostic. Age at death, sex and complete neuropathologically were provided. Autopsy tissue from human brains were collected with informed consent of patients or their relatives and approval of local institutional review boards. This study was approved by the University Health Network Research Ethics Board (Nr. 20-5258). Prior to inclusion in the study, a systematic neuropathological examination was performed following diagnostic criteria of neurodegenerative conditions and co-pathologies [20]. The contralateral hemisphere was sliced coronally at the time of autopsy and immediately flash frozen and stored at −80° C. Using a 4-mm brain tissue punch, a microdissection of the following regions was performed: anterior cingulate cortex, anterior cingulate white matter, frontal cortex, frontal white matter, putamen, globus pallidus, amygdala, hippocampus, temporal cortex, temporal white matter, substantia nigra, pons base, and cerebellar white matter. All the punches were stored in low binding protein tubes (Eppendorf), immediately flash frozen and stored at −80° C.

### Protein Extraction

For the PBS-soluble fraction, 40-50 mg of frozen microdissected tissue was thawed on wet ice and then immediately homogenized in 500 μl of PBS spiked with protease (Roche) and phosphatase inhibitors (Thermo Scientific) in a gentle-MACS Octo Dissociator (Miltenyi BioTec). The homogenate was transferred to a 1.5-ml low binding protein tube (Eppendorf) and centrifuged at 10,000 g for 10 min at 4° C., as previously described [21]. Then, the supernatant was collected and aliquoted in 0.5 mL low binding protein tubes (Eppendorf) to avoid excessive freeze-thaw cycles. Sarkosyl-insoluble material was extracted using 1 g of frozen brain tissue from three brain regions (cerebellum, putamen and frontal cortex) of individuals with MSA, as previously described [9]. A bicinchoninic acid protein (BCA) assay (Thermo Scientific) was performed to determine total protein concentration of all the aliquots.

### Histological Analysis

Four μm-thick formalin-fixed paraffin-embedded tissue sections containing the thirteen anatomical regions selected for microdissection (see above) were examined. In addition to Hematoxylin and Eosin-Luxol Fast Blue, the following mouse monoclonal antibodies were used for immunohistochemistry: anti aggregated α-synuclein (5G4; 1:4000; Roboscreen), nitrated anti-α-synuclein (Syn514; 1:2000; Biolegend), C-terminal truncated x-122 anti-α-synuclein (A15127A; 1:2000; Biolegend) and anti-phospho-α-synuclein (pSyn #64; 1:10000; FUJIFILM Wako Pure Chemical Corporation). To map co-pathology, we used the following mouse monoclonal antibodies: anti-tau AT8 (pS202/pT205; 1:1000; Thermo Scientific), anti-phospho-TDP-43 (pS409/410; 1:2000; Cosmo Bio), and anti-Aβ (6F/3D; 1:50; Dako). The DAKO EnVision detection kit, peroxidase/DAB, rabbit/mouse (Dako) was used to visualize the antibody staining. For the comparison of different α-synuclein antibodies, immunostained sections against nitrated, truncated, phosphorylated, and aggregated (5G4) α-synuclein were scanned using Tissuescope™ and were cropped with the HuronViewer™ (Huron). Images were taken from the exact same location of the putamen and cerebellum across the different antibodies. Initially, 100 immunoreactive oligodendrocytes from each antibody, region and case with visible nucleus were optically dissected using Photoshop. Using Image J, the minimum and maximum areas (px²) of the 100 inclusions were recorded. The density of black dots per unit of inclusions were measured. The amount of aggregated α-synuclein as well as the GCI and NCI burden were assessed using α-synuclein immunohistochemistry in the 13 brain regions above mentioned using the 5G4 staining. For semi-quantitative analyses, we used a 4-point scale:0, absent; 1, mild; 2, moderate; and 3, severe, as previously described [22].

### ELISA

Human α-synuclein Patho and total ELISAs kits (Roboscreen GmbH) were used according to manufacturer's protocol and as previously described [23].

### SDS-PAGE and Immunoblotting

Gel electrophoresis was performed using 4-12% or 12% Bolt Bis-Tris Plus gels (Thermo Scientific). Proteins were transferred to 0.45 μm polyvinylidene fluoride membranes for 60 min at 35 V. Proteins were crosslinked to the membrane via 0.4% (v/v) paraformaldehyde incubation in PBS for 30 min at room temperature, with rocking. Membranes were blocked for 60 min at room temperature in blocking buffer (5% (w/v) skim milk in 1×TBST (TBS and 0.05% (v/v) Tween-20) and then incubated overnight at 4° C. with primary antibodies diluted in blocking buffer. An antibody directed against amino acids 15-123 of the α-synuclein protein (BD Biosciences, 610786; 1:10,000 dilution) was used as primary antibody, as previously described [8,24]. Membranes were then washed three times with TBST and then incubated, for 60 min at room temperature, with horseradish peroxidase-conjugated secondary antibodies (Bio-Rad, 172-1011) diluted 1:10,000 in blocking buffer. Following another three washes with TBST, immunoblots were developed using Western Lightning enhanced chemiluminescence Pro (PerkinElmer) and imaged using X-ray film or the LiCor Odyssey Fc system.

### RT-QuIC Assay and Buffer Preparation

RT-QuIC reactions were performed in 384-well plates with a clear bottom (Nunc). Recombinant α-synuclein (rPeptide) was thawed from −80° C. storage, reconstituted in HPLC-grade water (Sigma) and filtered through a 100-kDa spin filter (Thermo Scientific) in 500-μL increments. All the reagents used for the reaction buffers were purchased from Sigma. 10 μL of the biological sample (5 μg of total protein from the PBS-soluble fraction) was added to wells containing 20 μL of the reaction buffer, 10 μL of 50 μM Thioflavin T and 10 μL of 0.5 mg/ml of monomeric recombinant α-synuclein. In every plate, positive (1 μg of α-synuclein preformed fibrils (rPeptide)) and negative (deonized water) controls were added. To ensure RT-QuIC reproducibility, a coefficient of variation no greater than 20% had be obtained when the positive control signals from different runs were compared. The plate was sealed and incubated at 37° C. in a BMG FLUOstar Omega plate reader with cycles of 1 min shaking (400 rpm double orbital) and 14 min rest. ThT fluorescence measurements (450+/−10 nm excitation and 480+/−10 nm emission, bottom read) was taken every 15 min for a period of 72 hours

### Conformational Stability Assays (CSA)

20 μL of 2× guanidine hydrochloride (GdnCl) stocks were added to an equal volume of the PBS-soluble fraction of the brain homogenates or RT-QuIC-derived α-synuclein fibrils to yield final GdnCl concentrations of 0, 1, 1.5, 2, 2.5, 3, 3.5 and 4 M, as previously described [24]. Briefly, PBS-soluble brain samples were incubated at room temperature with shaking for 120 min (800 RPM) before being diluted to 0.4 M GdnCl in PBS. Following high speed ultracentrifugation at 100,000×g for 60 min at 4° C. pellets were resuspended in 1×LDS loading buffer and boiled for 10 min. Levels of residual α-synuclein were determined by SDS-PAGE followed by immunoblotting, as above mentioned. Densitometry was performed using ImageJ, and values were normalized to the sample with the highest intensity, which was set at 100%. GdnCl₅₀ values, the concentration of GdnCl at which 50% of the aggregates are solubilized, were determined by nonlinear regression using the sigmoidal dose-response (variable slope) equation in GraphPad Prism, with the top and bottom values fixed at 100 and 0, respectively.

### Thermolysin (TL) Digestion

TL digestion was performed as previously described [8,24], with minor modifications. A concentration of 50 μg/mL of TL was added to PBS-soluble brain homogenates. RT-QuIC-derived α-synuclein fibrils were diluted into 1×PBS containing TL at a concentration of 5 μg/mL. Samples were incubated at 37° C. with continuous shaking (600 RPM) for 60 min. Digestions were halted with the addition of EDTA to final concentration of 2.5 mM, and samples were ultracentrifuged at 100,000×g for 60 min at 4° C. Supernatant was discarded and pellets resuspended in 1×LDS buffer and analyzed via SDS-PAGE followed by immunoblotting, as described above.

### Electron Microscopy

Aliquots (5 μl of α-synuclein fibril preparations) containing different RT-QuIC-derived α-synuclein fibrils were loaded onto freshly glow-discharged 400 mesh carbon-coated copper grids (Electron Microscopy Sciences) and adsorbed for 1 min. Once dry, grids were visualized using a Talos L120C transmission electron microscope (Thermo Fisher) using an acceleration voltage of 200 kV. Electron micrographs were recorded using an Eagle 4k×4k CETA CMOS camera (Thermo Fisher).

### Statistics

Statistical analyses were performed using GraphPad Prism (v.9) with a significance threshold of P=0.05. RT-QuIC relative fluorescence responses were also analyzed and plotted using the software GraphPad Prism (v.9). Comparisons were made using unpaired two-tailed t-tests and one-way ANOVA with Tukey's multiple comparisons test. Two-tailed Spearman r non-parametric correlations were used to correlate different variables obtained from single individuals using SPSS.

### Results

### Screening 168 RT-QuIC Conditions to Detect α-Synuclein Seeding in MSA

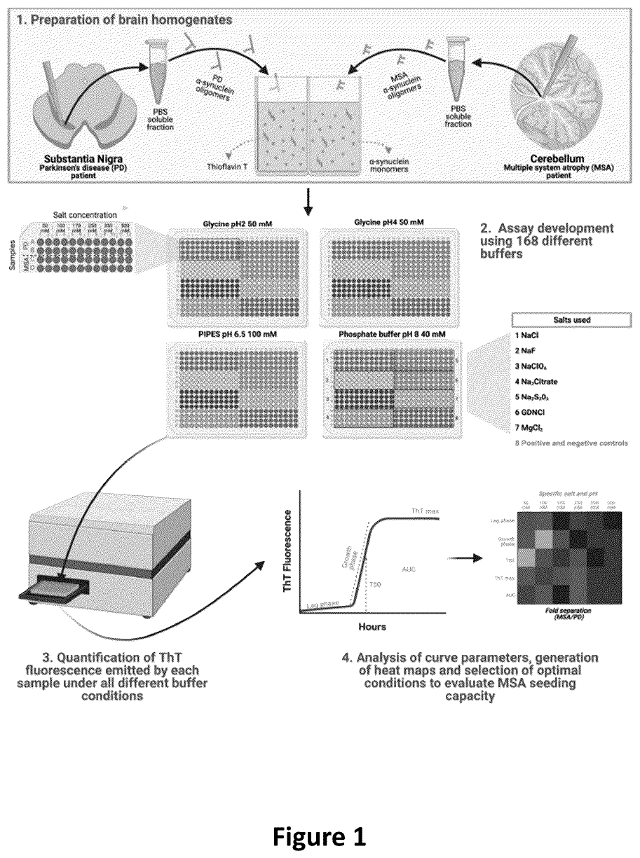

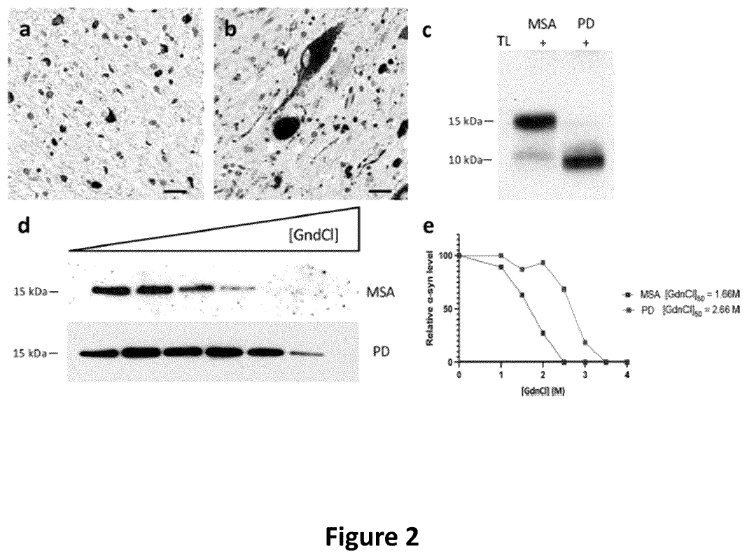

We hypothesized that changing the physicochemical factors that govern the in vitro amplification of amyloidogenic proteins would favor α-synuclein seeding in MSA. Based on the published success of using different ionic environments to enhance the sensitivity of different proteopathic seeding amplification assays [19], we conducted a systematic evaluation of 168 different reaction buffers, using an array of pH and salts, seeded with brain homogenates from one MSA and one PD patient (FIG. 1). We compared the effects of conducting the α-synuclein RT-QuIC in the presence of strongly (Citrate³⁻ and S₂O₃2⁻), moderately (F⁻ and Cl⁻) and weakly (ClO₄⁻) hydrated anions and cations (GdnCl and MgCl₂) at high (500 and 350 mM), medium (250 and 170 mM) and low (100 and 50 mM) concentrations using 4 different pH (pH2, pH4, pH6.5 and pH8). To seed the resulting 168 buffers, equal amounts (5 μg) of total brain protein were used per reaction. A region rich in Lewy body pathology, the substantia nigra, from a PD patient and a region enriched with GCIs, the cerebellar white matter, from a MSA patient were carefully microdissected using a 4-mm brain tissue punch and subsequently processed into crude homogenates, using PBS. In these two cases, conventional immunohistochemistry for disease-associated α-synuclein demonstrated abundant Lewy bodies and neurites in the SN of the PD patient and GCIs in the cerebellar white matter of the MSA patient (FIG. 2a-b). Next, we looked for further biochemical and structural differences in the α-synuclein brain homogenates. Preliminary studies were performed to compare the digestion of PBS soluble α-synuclein with proteinase K or with thermolysin. We found that thermolysin digestion offered a superior discriminative cleavage pattern between MSA and PD brain, that was also replicated when the RT-QuIC products were digested. All subsequent experiments therefore report the results of thermolysin digestion. The banding pattern of TL-resistant α-synuclein species in MSA was different from that in PD. While both brain extracts possessed a band at ˜10 kDa, the MSA extract displayed a prominent additional band at ˜15 kDa (FIG. 2c). Finally, using a CSA, that measures the differential abilities of protein aggregate strains to resist denaturation by GdnCl, we found that α-synuclein aggregates from the MSA brain extract were significantly less stable than those present in the PD brain extract (FIG. 2d-e).

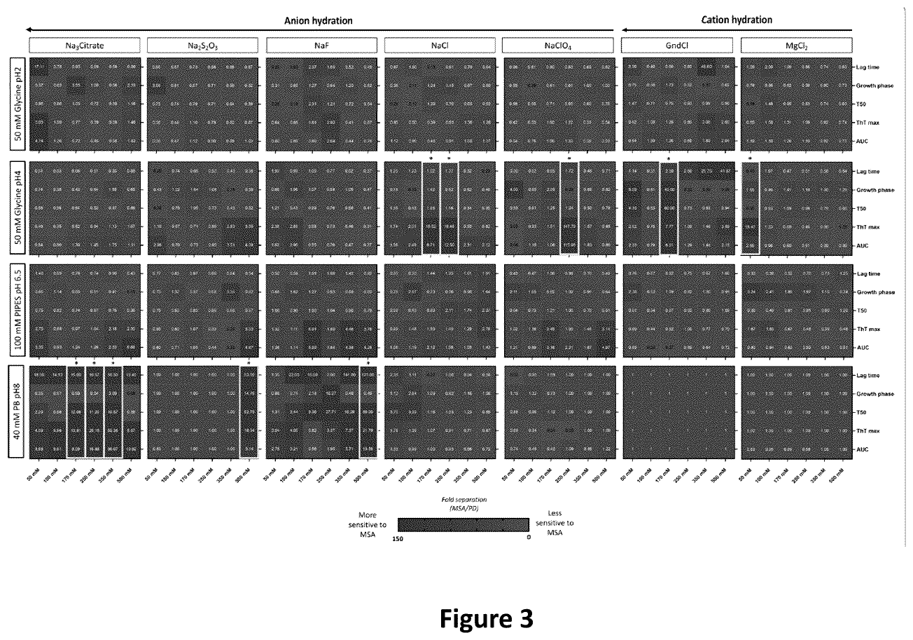

Following the characterization of the brain homogenates, and to identify the optimal assay conditions that favor MSA seeding activity, we seeded 168 different RT-QuIC reaction buffers using either 5 μg of MSA-derived or PD-derived PBS-soluble fraction. Four different RT-QuIC plates were ran in this phase (FIG. 1). Each condition was tested in quadruplicate and the same positive and negative controls were added in each plate. We then quantified 5 kinetic parameters from each reaction: lag time, growth phase, T50 (which corresponds to the time needed to reach 50% of maximum aggregation), ThT max or fluorescence peak, and area under the curve (AUC) of the fluorescence response. Each kinetic parameter was calculated using the mean of the values obtained by each quadruplicate. Importantly, we confirmed that no significant differences were observed in any of these 5 parameters between the positive and negative control samples run on all plates. Next, we calculated the fold separation of the 5 kinetic parameters examined by dividing the values obtained by the MSA and the PD brain homogenates, and generated heatmaps (FIG. 3). Larger numbers (shown in blue in FIG. 3) represent the conditions that showed a more favorable environment to detect MSA α-synuclein aggregation. When the fold separation was 1 (shown as deep red in FIG. 3), no discrimination between MSA and PD aggregation was found. Lower numbers (<1, shown as lighter red/purple in FIG. 3) represent the conditions more favorable to detect PD aggregation over MSA. Then, to select the conditions that were most favorable to detect MSA-derived α-synuclein aggregation,1 we defined the AUC as the seeding parameter of interest as it incorporates all the kinetic features of each aggregation reaction, including the speed and extent of aggregation. Our analysis revealed that 19/168 buffers were able to detect predominantly PD-derived α-synuclein aggregation, showing at least twofold difference between PD and MSA curves. 109/168 buffers showed no major differences in the main parameters describing the kinetic curve of the RT-QuIC when the MSA aggregation pattern was compared to PD. 40/168 buffers showed at least twofold difference between MSA-derived α-synuclein and PD-derived α-synuclein aggregation. From these 40 buffers, we carefully examined the parameters describing the kinetic curves from the 10 conditions that displayed the highest differences (data not shown). Interestingly, these buffers had a bimodal distribution where higher differences between MSA-derived α-synuclein and PD-derived α-synuclein aggregation were observed with low hydrated anions and cations when the reaction occurred at pH4, contrasting with the reactions performed at pH8 where the highly hydrated anions had the best performance (FIG. 3). Based on the raw values obtained with each condition, we selected two buffers: 50 mM glycine pH4 250 mM NaClO₄ (buffer 1) and 40 mM PB pH8 350 mM Na₃Citrate (buffer 2) for further analysis.

### Validation of the Two Optimal Conditions for Assessing α-Synuclein Seeding in MSA

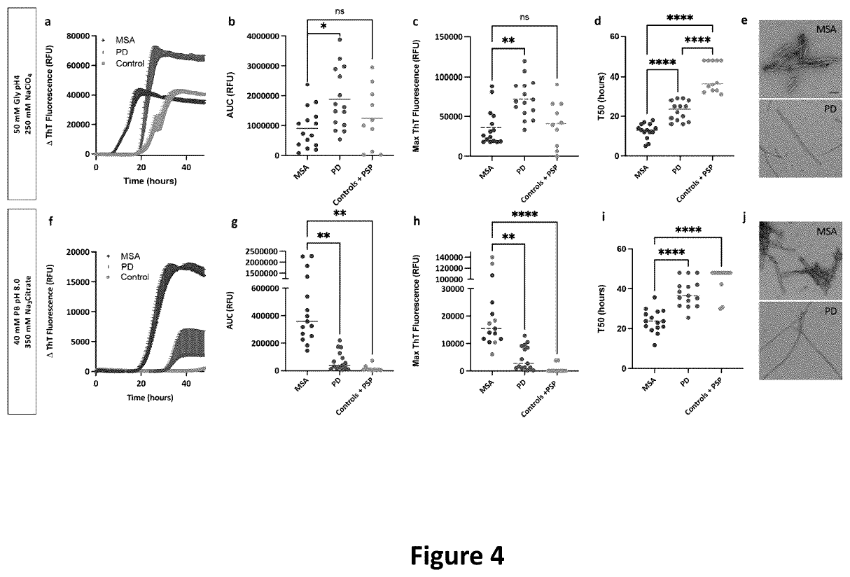

Having selected the two optimal buffers to detect MSA-derived α-synuclein seeding activity and differentiate it from PD-derived α-synuclein seeding activity, we proceeded to validate the results in a larger cohort of disease samples. We microdissected the substantia nigra of 15 subjects with MSA, 15 subjects with PD, 5 subjects with supranuclear progressive palsy and 5 controls and processed the samples into PBS soluble homogenates. All subjects had a detailed neuropathological examination (data not shown). To exclude potential differences, during the protein extraction, in α-synuclein aggregates recovery between MSA and PD subjects, we quantified the amount of total and aggregated α-synuclein using ELISAs. No differences either in the overall amount of total α-synuclein or in the aggregated α-synuclein were found between the two group of patients (data not shown). Equal amounts (5 μg) of total protein were used to seed the reactions from the 40 subjects, using the two buffer conditions, with all samples run in quadruplicate on the same plate. To illustrate the typical profile of α-synuclein RT-QuIC aggregation for samples of PD and MSA, we plotted data from one representative MSA, one PD and one control case (the median subject was used in each example curve) amplified under the buffer 1 condition (FIG. 4a). The maximum fluorescence and the AUC were consistently different for PD and MSA, with samples from patients with MSA consistently aggregating faster while always reaching a lower fluorescence plateau than samples from patients with PD (FIG. 4b-c). When we plotted data from the same cases but amplified under the buffer 2 condition, we observed that samples from patients with MSA still aggregated faster but in contrast, using this buffer, they reached a higher fluorescence plateau than those from patients with PD (FIG. 4f). Furthermore, significant differences in the maximum fluorescence and the AUC were observed between PD and MSA patients (FIG. 4g-h). To test whether the amount of ThT bound to the MSA and PD fibrils depends on the physicochemical factors of the buffer, we investigated if the end products from the RT-QuIC assay amplified under the two conditions showed structural differences when examined using electron microscopy (FIG. 4e, j). Interestingly, and despite the striking differences in the ThT kinetics shown by the two buffers, under both conditions the fibrils derived from the PD samples consistently showed a thin and straight morphology, while the fibrils derived from the MSA samples were consistently thicker and twisted. However, we were not able to determine whether the PD or the MSA fibrils generated under different conditions presented structural differences. When we compared the kinetics shown by the PD and MSA patient samples with a cohort of controls and supranuclear progressive palsy patient samples without immunohistochemically detectable α-synuclein co-pathology, the MSA samples did not show significant differences in either the maximum fluorescence or in the AUC compared to controls or supranuclear progressive palsy subjects amplified under the buffer 1 condition (FIG. 4b-c). However, the MSA samples showed faster aggregation (T50) than the control and supranuclear progressive palsy group (FIG. 4d). When the buffer 2 condition was examined, we found an optimal distinction between MSA and PD and maximized the kinetic separation from controls and supranuclear progressive palsy subjects both in the maximum fluorescence, T50 and in the AUC (FIG. 4g-i). Therefore, the buffer 2 or the 40 mM PB pH8 350 mM Na₃Citrate condition was selected as the optimal working buffer to study MSA α-synuclein seeding in RT-QuIC assays.

### Characterization of the RT-QuIC End-Products

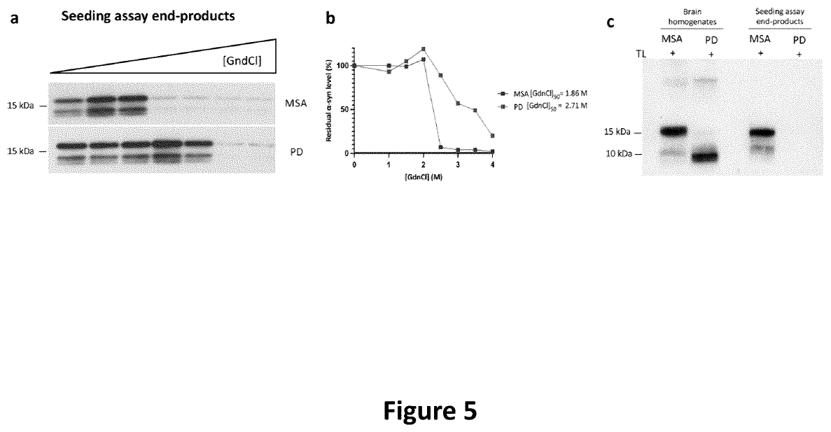

To gain further insight regarding the structures of the aggregates that were amplified from patients with PD or with MSA, we analyzed the buffer 2 RT-QuIC end-products by CSA and TL digestion. For these experiments, we examined the RT-QuIC end-products derived from the PD and MSA subjects used to seed the assays of the screening phase. As previously mentioned, under the CSA, the aggregates present in the MSA brain homogenate were significantly less stable following exposure to GdnCl than those found in the PD brain homogenate. This difference in conformational stability was preserved in the epitope (amino acids 15-123) used to probe the structure following the in-vitro amplification by RT-QuIC, and the end-products derived from the MSA brain were less stable than the PD ones (FIG. 5a-b). When the banding pattern of TL-resistant α-synuclein species was examined, we found that the MSA homogenate and the MSA-derived RT-QuIC end-products possessed a band at ˜10 kDa and a prominent additional band at ˜15 kDa (FIG. 5c). However, when the PD samples were examined, the brain homogenate showed a band at ˜10 kDa, but the α-synuclein present in the PD RT-QuIC end-product was not TL-resistant. These results, together with the ultrastructural data, show that, under our assay conditions, RT-QuIC reaction products seeded from MSA α-synuclein fibrils are conformationally distinguishable from RT-QuIC reaction products seeded from PD-derived fibrils.

### MSA Patients Present Distinct α-Synuclein Seeding

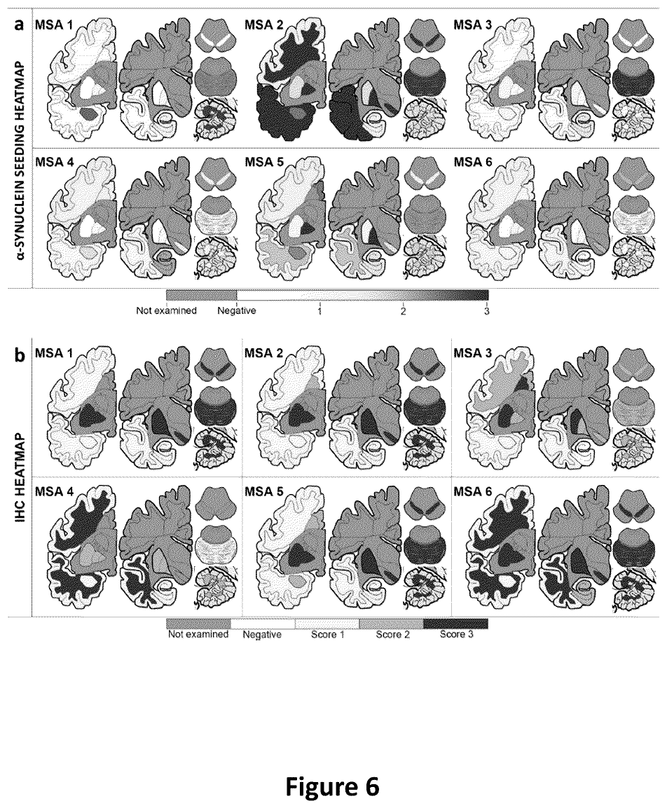

In addition to the observed differences in the AUC and ThT max values among the different groups of patients examined, when the individual values of the 15 MSA subjects were examined, we observed up to tenfold differences in AUC and ThT max values among MSA samples (FIG. 4f-g). Based on the data derived from the substantia nigra samples, the subjects with MSA included in our study were further subtyped into 3 groups: high, intermediate and low seeders, according to their ability to misfold monomers of recombinant α-synuclein in vitro. To test the hypothesis that heterogeneity in the α-synuclein seeding exists in MSA, from our initial cohort of 15 MSA patients, we selected six subjects according to their α-synuclein seeding behavior (FIG. 4g) where two were classified as low seeders (subjects MSA 1 and MSA 6), two as intermediate seeders (subjects MSA 3 and MSA 4) and two as high seeders (subjects MSA 2 and MSA 5). To assess whether these distinctions could be due to differences in seed concentration or seed characteristics, we performed end-point dilutions of the cerebellar PBS-soluble fraction from two MSA cases classified as high and low seeders (data not shown). Regardless of the amount of brain homogenate used to seed the RT-QuIC reaction, the seeding activity of the misfolded α-synuclein from the high seeder case was more potent than the one found in the low seeder case (data not shown). Then, the α-synuclein seeding behavior was then assessed in 13 different brain regions from these subjects. From each case, we micro-dissected and subsequently prepared a PBS-soluble extract from the following regions: anterior cingulate cortex, anterior cingulate white matter, frontal cortex, frontal white matter, putamen, globus pallidus, amygdala, hippocampus, temporal cortex, temporal white matter, substantia nigra, pons base and cerebellar white matter (data not shown). All samples were run in quadruplicate, and the RT-QuIC curve obtained by each reaction was evaluated as a measure of the α-synuclein seeding behavior (data not shown). The AUC values were converted into scores ranging from 0 to 3. With these values we generated a heatmap of α-synuclein seeding, to illustrate the differences found between brain regions and patients (FIG. 6a). Interestingly, when the mean AUC values from all the regions included were analyzed, the two patients categorized as high seeders had significantly higher AUC values than the subjects categorized as intermediate (p<0.0001) or low (p<0.0001) seeders. When the different brain regions were examined individually, the pons base showed the highest α-synuclein seeding. Surprisingly, the cerebellar white matter and the putamen were the regions where the lowest α-synuclein seeding was observed. When the different cortices were examined, we found that the frontal and temporal cortex displayed a medium seeding, while the cingulate cortex had higher seeding. A higher seeding was consistently observed in the white matter of the three cortices when compared to the grey matter. Overall, the hippocampus displayed a medium seeding, while the α-synuclein present in globus pallidus, SN and the amygdala had a higher seeding.

### Biochemical and Neuropathological Characterization of α-Synuclein

The factors that underlie the ability of α-synuclein from different patients with MSA to drive higher versus lower seeding are presently unknown. To investigate this further we conducted a biochemical examination of the α-synuclein derived from the brain homogenates coupled with a detailed immunohistochemical mapping of α-synuclein deposition in corresponding samples. First, we quantified the amount of total and aggregated α-synuclein using ELISAs and correlated these measures with their α-synuclein seeding behavior. MSA cases had 0.6-4 ng of total α-synuclein per mg of tissue in the PBS soluble fraction. No differences in the overall amount of α-synuclein were found between high, low and intermediate seeders. The regions where total α-synuclein was more abundant were the amygdala and the frontal cortex, whereas the cerebellar white matter and the pons were the regions where the α-synuclein was less expressed (data not shown). The amount of aggregated α-synuclein was quantified using the α-synuclein Patho ELISA. MSA cases had 3-362 μg of aggregated α-synuclein per mg of tissue in the PBS soluble fraction. Interestingly, the amount of aggregated α-synuclein was significantly higher (p=0.0165) in the low seeders compared to the high seeders. The regions that had a higher burden of aggregated α-synuclein were the temporal and the anterior cingulate cortex, whereas, as occurred with the total α-synuclein levels, the cerebellar white matter and the pons were the regions where the least aggregated α-synuclein was found (data not shown). The total amount of α-synuclein in the PBS-soluble fraction did not correlate with either the amount of aggregated α-synuclein (p=0.171) or with the α-synuclein seeding (p=0.648). However, the levels of aggregated α-synuclein were negatively correlated with the α-synuclein seeding (p=0.035, r=−0.241). These data, together with the striking seeding activity in soluble fractions from brain regions without noticeable accumulations of α-synuclein, lead us to test whether the heterogeneity between regions and patients was also maintained if larger, sarkosyl-insoluble (SI), α-synuclein aggregates were used to seed the RT-QuIC assay. To test this hypothesis, we extracted α-synuclein SI aggregates from the cerebellum, putamen and frontal cortex from one MSA case classified as a low seeder (MSA 1) and one MSA case categorized as a high seeder (MSA 2). Equal amounts (5 μg) of total protein were used to seed all the reactions and quadruplicates from these samples together with their corresponding PBS-soluble fractions were evaluated. Furthermore, owing to the insoluble nature of the SI aggregates and possible inaccessibility of the fibril ends, we also included sonicated SI samples. When the RT-QuIC curves were evaluated, no significant differences between regions were observed in the SI aggregates. However, the SI aggregates from the three brain regions of the high seeder (MSA 2) aggregated faster and reached a higher fluorescence plateau than the SI aggregates from the low seeder (MSA 1, data not shown). Regardless of the region or the patient examined, the SI aggregates promoted a faster aggregation and reached a higher fluorescence plateau than the α-synuclein aggregates present in the corresponding PBS-soluble fraction (data not shown). The same pattern was observed when the differences in the α-synuclein aggregation kinetics were examined between SI aggregates with and without sonication, where the sonicated SI aggregates promoted a faster aggregation and reached a higher fluorescence plateau than the non-sonicated (data not shown).

Finally, we aimed to evaluate whether the heterogeneity of α-synuclein seeding was reflected in immunohistological findings in the brains of the same subjects. First, we performed an epitope mapping to compare which α-synuclein antibody was able to detect the most MSA α-synuclein pathology in formalin-fixed paraffin-embedded tissue sections. Using consecutive sections from the putamen and the cerebellum from our cohort of 6 MSA patients, we performed morphometric analysis using four different α-synuclein antibodies: the 5G4 conformational antibody that recognizes aggregated forms of α-synuclein, the pSyn #64 that recognizes Ser129 phosphorylated α-synuclein, and two antibodies against the truncated (C-terminal cleaved) and nitrated forms of α-synuclein (clones A15127A and Syn514, respectively) (data not shown). The conformational 5G4 antibody consistently exhibited more α-synuclein pathology in all the cases and regions examined followed by the two antibodies that recognized Ser 129 phosphorylated α-synuclein, and truncated α-synuclein (data not shown). Thus, using the 5G4 antibody, a semi-quantitative analysis of the α-synuclein aggregation burden was performed, the result of which are presented as a heatmap (data not shown). The cerebellum and the putamen were the regions with the highest aggregated α-synuclein, with the SN, the pons base and the globus pallidus also exhibiting a high burden of aggregated α-synuclein. The grey matter from the temporal and frontal cortex were the regions where less amount of aggregated α-synuclein was found. In addition to the semi-quantitative analysis of the total burden of aggregated α-synuclein, the burden of GCIs and neuronal cytoplasmic inclusions (NCIs) was also examined (data not shown). As expected, the presence of NCIs was less frequent than the presence of GCIs. The pons base, SN and putamen, followed by the hippocampus and the anterior cingulate cortex, were the regions where more NCIs were evident. When the GCIs burden was evaluated, the putamen and cerebellum, followed by the globus pallidus and the SN were the regions with more abundant GCIs, while the hippocampus and the frontal cortex has less abundant GCIs. Surprisingly, there was no significant difference in the burden of GCIs, NCIs or aggregated α-synuclein between high, low and intermediate seeders, and no correlation was found between the burden of GCIs (p=0.957) or NCIs (p=0.252) with the α-synuclein seeding.

Here we show that the physicochemical factors that govern the in vitro amplification of α-synuclein can be tailored to generate strain-specific reaction buffers for use in RT-QuIC. Using this novel approach, we have generated a streamlined RT-QuIC assay that 1) is able to measure the α-synuclein seeding of 96 MSA samples, run in quadruplicate, in less than 48 hours; 2) requires the use of a minimal amount of commercially available recombinant α-synuclein monomer (5 μg) per well; 3) requires the use of a minimal amount of brain material (5 μg); 4) generates RT-QuIC-derived α-synuclein fibrils that are conformationally distinct between patients with different synucleinopathies; and 5) is capable of subtyping MSA brains according to their α-synuclein seeding behavior.

MSA is clinically and pathologically heterogeneous, and although the distribution of GCIs and NCIs might correlate with the predominant clinical features [25,26], the burden of α-synuclein inclusions does not fully explain the differences in clinical presentation and rate of disease progression exhibited in MSA. In recent years, mounting evidence highlights the important role of amyloidogenic proteins in soluble brain fractions to seed pathologic aggregation, in a prion-like manner, that might contribute to clinical heterogeneity seen in patients with neurodegenerative diseases[21]. The capacity of misfolded α-synuclein to template monomeric α-synuclein has been exploited by several groups to generate an RT-QuIC assay able to measure seeding kinetics of α-synuclein in a range of samples in PD [14,17,27-29]. However, the reliable detection of α-synuclein seeding in MSA derived samples has been restricted to a couple of studies [18,30]. To identify the optimal conditions to evaluate the α-synuclein seeding in MSA-derived samples, we performed a comprehensive analysis of 168 different reaction buffers, covering an array of pH and salts. We then validated the two conditions that conferred the optimal ability to discriminate between PD and MSA-derived samples in a larger cohort of neuropathologically confirmed cases. Although the same samples were used to seed the RT-QuIC reactions, the kinetics of the curves obtained using these two different buffers were significantly divergent, suggesting that the accessibility or the interaction of the ThT with α-synuclein from MSA and PD can be modulated by the type of salt, pH or ionic strength of the reaction buffer [18,19,31]. We selected the 40 mM PB pH8 350 mM Na₃Citrate buffer, as the optimal condition with which to evaluate α-synuclein seeding in MSA and further demonstrated that the RT-QuIC-derived MSA-fibrils maintained the biochemical properties of the MSA aggregates [24] used to seed the reaction.

Our multi-region mapping of the α-synuclein seeding across 13 different brain regions in MSA revealed that the pons base had the highest seeding. Surprisingly, α-synuclein extracted from two of the most-affected regions in MSA, the cerebellum and the putamen, exhibited the lowest seeding observed. A possible explanation for these results is that the analysis of the α-synuclein seeding was performed using the PBS soluble fraction. It is plausible that the seeding activity of α-synuclein could diminish over time as the more soluble seeding competent α-synuclein species, present at earlier stages of disease, become sequestered in larger insoluble aggregates at later stages of the disease. This could then result in reduced seeding of α-synuclein extracted from regions affected earlier in the disease course [21,32]. This possibility led us to compare the seeding behavior of SI α-synuclein aggregates and the α-synuclein seeds present in the PBS soluble fraction in different brain regions. When the α-synuclein seeding was compared, regional differences were only observed using the PBS soluble fraction and not using the SI aggregates. However, regardless of the region or the patient examined, the SI aggregates consistently promoted a faster aggregation than the α-synuclein seeds present in the corresponding PBS-soluble fraction, suggesting that there are more competent α-synuclein species in the SI fraction. Our findings support earlier observations that modifications and solubility of α-synuclein in MSA may be more widespread than obvious histopathology [33,34] and might be different between distinct synucleinopathies [34]. Future experiments will investigate the curious finding that PBS soluble α-synuclein is the major contributor to the observed regional differences in the seeding behavior in MSA. Interestingly, and irrespective of the differences found between the SI and the soluble fraction, striking differences in the seeding between patients were also observed.

Our study is subject to some limitations. Our data clearly demonstrate that the selection of the right conditions to conduct the amplification of the seeds is of major relevance. As a field, collectively our understanding of the optimal in vitro physicochemical factors with which specific seed conformers can propagate is still in its infancy. Using suboptimal conditions, an absence of seeding activity could reflect the absence of seeds, but also might reflect an inability of the specific RT-QuIC conditions to support the amplification of those particular seeds [35].

Our findings pave the way for future studies to address the molecular mechanisms underlying the ability of α-synuclein from different patients with MSA to drive higher versus lower seeding. Indeed, our results support a model whereby the heterogeneity observed both between different brain regions and between different MSA patients might be due to differences in the cellular environment [6,8] and to the presence of posttranslational modifications [9] and other cofactors, such as p25α [36], that may confer a selective pressure for one α-synuclein conformation over another [6].

This study demonstrates that the physicochemical factors that govern the in vitro amplification of α-synuclein can be tailored to generate strain-specific reaction buffers for use in RT-QuIC. Using this novel approach, we have found unexpected differences in seed-competent α-synuclein across a cohort of neuropathologically comparable MSA brains. Understanding the relationship between the seeding differences and biological activity of α-synuclein will be critical for the future subclassification of MSA, that should go beyond the conventional clinical and neuropathological phenotyping and consider the structural and biochemical heterogeneity of α-synuclein present in these patients, which will allow the developing of new personalized therapeutic strategies for this devastating disease. Furthermore, a deeper understanding of how physicochemical factors influence the aggregation of different α-synuclein strains will provide support for the development of vitally needed, rapid and sensitive in vivo assays for the diagnosis of MSA and other synucleinopathies.

### Example 2

### Methods and Materials

### Participants

Participants were recruited from the Toronto Western Hospital Movement Disorders Centre, University Health Network Research Ethics Board approved study 20-5558. All patients met strict diagnostic criteria for PD [44], MSA [45] or PSP [46].

TABLE 1
Cohort characteristics

| Disease (n) | PD (13)^{a} | | MSA (10)^{a} | | PSP (7) | | Controls (20) | |
|---|---|---|---|---|---|---|---|---|
| Sex | N | (%) | N | (%) | N | (%) | N | (%) |
| Women | 5 | (39%) | 9 | (90%) | 3 | (43%) | 9 | (45%) |
| Men | 8 | (62%) | 1 | (10%) | 4 | (57%) | 11 | (55%) |
| | mean | (SD) | mean | (SD) | mean | (SD) | mean | (SD) |
| Age-at-assessment | 61 | (10) | 58 | (8) | 70 | (6) | 67 | (7) |
| Age-at-onset | 53 | (12) | 54 | (9) | 65 | (5) | n/a | |
| Disease duration | 8 | (6) | 3 | (1) | 5 | (3) | n/a | |
| | N | (%) | N | (%) | N | (%) | N | (%) |
| Family history of parkinsonism | 3 | (23%) | 3 | (30%) | 1 | (14%) | 6 | (30%) |
| Medications | N | (%) | N | (%) | N | (%) | N | (%) |
| Levodopa | 10 | (77%) | 7 | (70%) | 4 | (57%) | n/a | |
| MAOBI | 2 | (15%) | 0 | (0%) | 0 | (0%) | n/a | |
| Dopamine agonists | 7 | (54%) | 1 | (10%) | 0 | (0%) | n/a | |
| COMTI | 0 | (0%) | 0 | (0%) | 0 | (0%) | n/a | |
| Anticholinergics | 0 | (0%) | 0 | (0%) | 0 | (0%) | n/a | |
| Amantadine | 2 | (15%) | 4 | (40%) | 3 | (43%) | n/a | |
| Levodopa equivalent dose (LEDD) | mean | (SD) | mean | (SD) | mean | (SD) | mean | (SD) |
| | 844 | (548) | 554 | (394) | 186 | (243) | n/a | |
| First symptom | N | (%) | N | (%) | N | (%) | N | (%) |
| Tremor | 9 | (69%) | 1 | (10%) | 0 | (0%) | n/a | |
| Bradykinesia | 5 | (39%) | 3 | (30%) | 0 | (0%) | n/a | |
| Rigidity | 8 | (62%) | 2 | (20%) | 0 | (0%) | n/a | |
| Writing difficulties | 0 | (0%) | 1 | (10%) | 0 | (0%) | n/a | |
| Gait problems | 1 | (8%) | 1 | (10%) | 1 | (14%) | n/a | |
| Early falls | 0 | (0%) | 0 | (0%) | 3 | (43%) | n/a | |
| Postural instability | 1 | (8%) | 3 | (30%) | 1 | (14%) | n/a | |
| Coordination problems | 0 | (0%) | 1 | (10%) | 0 | (0%) | n/a | |
| Diplopia | 0 | (0%) | 0 | (0%) | 1 | (14%) | n/a | |
| Speech problems | 0 | (0%) | 0 | (0%) | 1 | (14%) | n/a | |
| Motor complications | N | (%) | N | (%) | N | (%) | N | (%) |
| Dyskinesia | 5 | (39%) | 2 | (20%) | 0 | (0%) | n/a | |
| Dystonia | 6 | (46%) | 4 | (40%) | 1 | (14%) | n/a | |
| Freezing of gait | 3 | (23%) | 4 | (40%) | 1 | (14%) | n/a | |
| Atypical clinical features | N | (%) | N | (%) | N | (%) | N | (%) |
| | 0 | (0%) | 0 | (0%) | 1 | (14%)* | 0 | (0%) |

Abbreviations: N: number, MAOBI: Monoamine Oxidase type B inhibitors, COMTI: Catechol o-Methyltransferase inhibitors, LEDD: Levodopa equivalent daily dose. * This patient had all the features of classic PSP-RS (fulfilling the new MDS-PSP diagnostic criteria for “probable PSP-RS”³) with no other clinical features supportive of MSA or PD with the exception of additional minor delusions, memory issues and evidence of amygdala thinning on MRI (these did not fulfill MDS-PSP exclusionary criteria³). These features could suggest the possibility of additional alpha-synuclein co-pathology.

### Sample Collection

One 4-mm punch biopsy was collected from the cervical 07 paravertebral area. Biopsies were rinsed in ice-cold phosphate buffered saline (PBS) spiked with protease inhibitor (complete EDTA-free Protease Inhibitor Cocktail, Roche), were blotted dry, transferred to a clean tube and frozen at −80° C. within 1 hour of collection. Blood serum was collected according to standardised procedures. 15 minutes after collection, serum was centrifuged for 15 minutes (1500×G) at 4° C. prior to aliquoting and storage at −80° C. All assays were performed blinded to the diagnosis of the patient or control.

### Real-Time Quaking-Induced Conversion Assay

Skin samples were washed three times in cold PBS, minced and homogenized at 10% (w/v) using a gentle-MACS Octo Dissociator (Miltenyi BioTec) in cold PBS spiked with phosphatase inhibitors (Thermo Scientific). For the RT-QuIC assay 4 μL of each sample was added to wells containing 20 μL of reaction buffer (40 mM PB, pH 8, 350 mM Na₃Citrate), 10 μL of 50 μM Thioflavin T and 10 μL of 0.5 mg/mL monomeric recombinant K23Q aSyn (Impact Biologicals) in a clear bottomed 384-well plate (SigmaAldrich). Each patient's sample was run in quadruplicate. The plate was sealed and incubated at 42° C. in a BMG FLUOstar Omega plate reader (BMG Labtech) with cycles of 1 min shaking (400 rpm double orbital) and 1 min rest for a period of 24 hours. ThT fluorescence measurements (450+/−10 nm excitation and 480+/−10 nm emission) were taken every 45 min. The cutoff value for defining the positive aSyn seeding activity was 20,000 (relative fluorescence units [rfu]) based on the mean aSyn seeding activity of control at 12 hours. At least 2 of 4 replicate wells had to cross this threshold with a lag phase of less than 13 hours in order for a sample to be considered positive.

### Neurofilament Light Chain

Serum NfL levels were measured using a commercial assay on an SR-X Single molecule array (Simoa) instrument (Quanterix, Lexington, MA), as described in detail previously [47]. Calibrators (neat) and samples (in a 4-fold dilution) were run as triplicates and duplicates respectively.

### Data Analysis

The investigators processing the samples (IMV and NPV), performing and analyzing the RT-QuIC (IMV), and measuring the serum NfL levels (AV and CA) were blinded to the clinical diagnosis. Once all the assays were completed, the results were sent to AEL and GGK for unblinding.

### Results

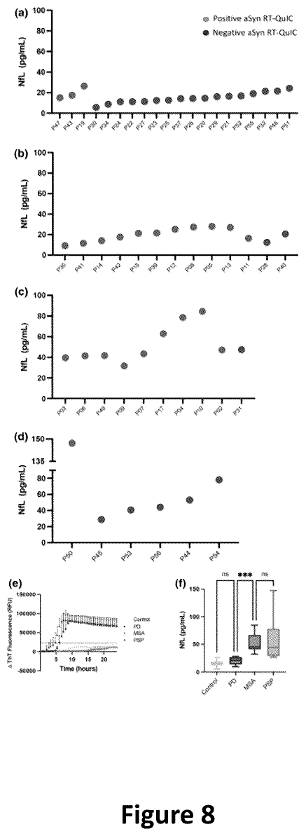

Here, we have employed our novel assay to assess aSyn seeding activity using a single 4-mm cervical skin biopsy from clinically diagnosed patients with MSA, PD, progressive supranuclear palsy (PSP) and healthy controls. All analyses were conducted blinded to the clinical diagnosis. Cutaneous aSyn RT-QuIC was positive in 9/10 patients with MSA, 11/13 patients with PD, 3/20 healthy controls and 1/7 patients with PSP (FIG. 8a-e), achieving a combined sensitivity of 87% and specificity of 85% for synucleinopathies vs non-synucleinopathies. However, the assay was not able to clearly distinguish MSA from PD based on seeding kinetics alone. Thus, based on the published success of circulating neurofilament light chain (NfL) as a potential early marker to distinguish patients with PD from those with atypical parkinsonian disorders [42], we analyzed the serum NfL concentration in all subjects (blinded to diagnosis) using an ultrasensitive detection system, the single molecule array (SIMOA). Serum NfL was significantly higher in patients with MSA than in patients with PD (p=0.0004, FIG. 1f), with no differences in circulating NfL found between controls and PD patients (p=0.9281, FIG. 8f) or between PSP and MSA patients (p=0.8019, FIG. 1f).

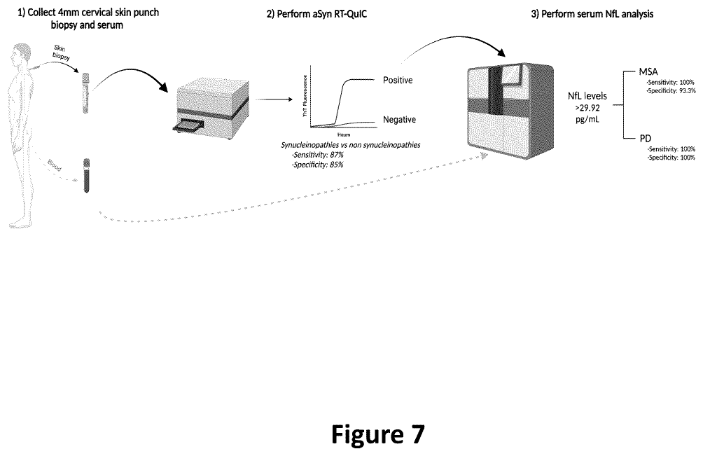

This is the first report to detect cutaneous aSyn seeding activity in both MSA and PD with high sensitivity and specificity compared to individuals with PSP and healthy controls (of note, the one positive PSP patient has additional clinical features that might suggest the presence of aSyn co-pathology [43]). Thus, a positive skin RT-QuIC was highly sensitive and specific for a synucleinopathy and the second step NfL assay differentiated MSA from PD (FIG. 7).

Indeed, we have consciously designed a streamlined assay that can be easily replicated and utilized by others, and we hope our work encourages replication in independent cohorts. The future application of our 2-step diagnostic approach in the screening process for clinical trials has the potential to ensure that trials for therapeutics targeting aSyn recruit individuals with a positive cutaneous aSyn RT-QuIC (and distinguish MSA from PD as necessary).

### Example 3

Despite recent advances in the detection of misfolded aSyn from peripheral sources as a reliable biomarker for the diagnosis of synucleinopathies, most assays rely on the examination of CSF, which poses an important technical/practical challenge for wide-scale clinical implementation. To overcome previous limitations, we describe a new method for extracting neuron, astroglial and oligodendroglia-derived pathological α-synuclein from extracellular vesicles (EVs) isolated from the blood.

EVs are lipid-bound vesicles secreted by cells into the extracellular space [48]. The three main subtypes of EVs are microvesicles, exosomes, and apoptotic bodies, which are differentiated based upon their biogenesis, release pathways, size, content, and function. The content, or cargo, of EVs consists of lipids, nucleic acids, and proteins [48]. Interestingly, EVs are discussed as players in the cell-to-cell spreading of misfolded proteins across the brain in neurodegenerative diseases [49]. This, together with the fact that EVs can cross the blood-brain barrier, and the availability of surface markers to capture exosomes of neuronal, glial and astrocytic origin [49], has made EVs an ideal candidate for a non-invasive source of brain-derived misfolded amyloidogenic proteins.

### Isolation Protocol

To isolate circulating EVs 10 millilitres of human blood is collected. Once the plasma and the serum are isolated from the whole blood, the EVs are extracted using from 100 microlitres to 5 millilitres of serum and/or plasma. The EVs can be extracted using one or a combination of the following techniques: differential ultracentrifugation, density gradient centrifugation, membrane particle precipitation-based, size exclusion chromatography, ultrafiltration coupled with size exclusion chromatography, affinity membrane-based column chromatography, immunological separation, coated magnetic beads with antibodies, lipid nanoprobe system, venceremin, or using microfluidic devices, or filtration systems.

After that, the isolation of EVs is confirmed by evaluating the expression of CD63, CD9, CD81, CD41a by ELISAs, western blotting and/or flow cytometry. Further characterization of their size and morphology is achieved by negative-stain transmission electron microscopy as well as dynamic light scattering, nanoparticle tracking analysis and tunable resistive pulse sensing.

After confirming that a uniform EVs population is obtained, neuron, astrocytes and oligodendroglia-derived EVs are isolated using neuronal cell surface markers (NCAM-L1, GluR2/3, SNAP25 and the like) astrocytes cell surface markers (GFAP, GLAST, GLUL, and the like) and oligodendroglia cell surface markers (PLP, CNP, MBP, MOG and the like) through immunoprecipitation and or flow cytometry.

After that, pure populations of neuron, astrocytes and oligodendroglia-derived extracellular vesicles are obtained and can be used as a biological sample for the RT-QuIC assay.

Although preferred embodiments of the invention have been described herein, it will be understood by those skilled in the art that variations may be made thereto without departing from the spirit of the invention or the scope of the appended claims. All documents disclosed herein, including those in the following reference list, are incorporated by reference.

### REFERENCE LIST

1. Gilman S, Wenning G K, Low P A, Brooks D J, Mathias C J, Trojanowski J Q, et al. Second consensus statement on the diagnosis of multiple system atrophy. Neurology. 2008; 71:670-6.
2. Graham J G, Oppenheimer D R. Orthostatic hypotension and nicotine sensitivity in a case of multiple system atrophy. J Neurol Neurosurg Psychiatry. 1969; 32:28-34.
3. Papp M I, Kahn J E, Lantos P L. Glial cytoplasmic inclusions in the CNS of patients with multiple system atrophy (striatonigral degeneration, olivopontocerebellar atrophy and Shy-Drager syndrome). J Neurol Sci. 1989; 94:79-100.
4. Grazia Spillantini M, Anthony Crowther R, Jakes R, Cairns N J, Lantos P L, Goedert M. Filamentous α-synuclein inclusions link multiple system atrophy with Parkinson's disease and dementia with Lewy bodies. Neurosci Lett. 1998; 251:205-8.
5. Peelaerts W, Bousset L, Van der Perren A, Moskalyuk A, Pulizzi R, Giugliano M, et al. α-Synuclein strains cause distinct synucleinopathies after local and systemic administration. Nature [Internet]. 2015 [cited 2017 Aug. 17]; 522:340-4. Available from: http://www.nature.com/doifinder/10.1038/nature14547
6. Holec S A M, Woerman A L. Evidence of distinct α-synuclein strains underlying disease heterogeneity. Acta Neuropathol [Internet]. Springer Berlin Heidelberg; 2020; Available from: https://doi.org/10.1007/s00401-020-02163-5
7. Woerman A L, St6 hr J, Aoyagi A, Rampersaud R, Krejciova Z, Watts J C, et al.
Propagation of prions causing synucleinopathies in cultured cells. Proc Natl Acad Sci USA [Internet]. 2015; 112:E4949-58. Available from: http://www.ncbi.nlm.nih.gov/pubmed/26286986
8. Peng C, Gathagan R J, Covell D J, Medellin C, Stieber A, Robinson J L, et al. Cellular milieu imparts distinct pathological α-synuclein strains in α-synucleinopathies. Nature [Internet]. 2018; 557:558-63. Available from: http://www.nature.com/articles/s41586-018-0104-4
9. Schweighauser M, Shi Y, Tarutani A, Kametani F, Murzin A G, Ghetti B, et al. Structures of α-synuclein filaments from multiple system atrophy. Nature [Internet]. Springer U S; 2020; 585:464-9. Available from: http://dx.doi.org/10.1038/s41586-020-2317-6
10. Guerrero-Ferreira R, Kovacik L, Ni D, Stahlberg H. New insights on the structure of alpha-synuclein fibrils using cryo-electron microscopy. Curr Opin Neurobiol [Internet]. Elsevier Ltd; 2020; 61:89-95. Available from: https://doi.org/10.1016/j.conb.2020.01.014
11. Ferreira N do C, Caughey B. Proteopathic Seed Amplification Assays for Neurodegenerative Disorders. Clin Lab Med [Internet]. Elsevier Inc; 2020; 40:257-70. Available from: https://doi.org/10.1016/j.cll.2020.04.002
12. Schmitz M, Cramm M, Llorens F, Müller-Cramm D, Collins S, Atarashi R, et al. The real-Time quaking-induced conversion assay for detection of human prion disease and study of other protein misfolding diseases. Nat Protoc. 2016; 11:2233-42.
13. Bongianni M, Orrù C, Groveman B R, Sacchetto L, Fiorini M, Tonoli G, et al. Diagnosis of human prion disease using real-time quaking-induced conversion testing of olfactory mucosa and cerebrospinal fluid samples. JAMA Neurol. 2017; 74:155-62.
14. Manne S, Kondru N, Jin H, Anantharam V, Huang X, Kanthasamy A, et al. α-Synuclein real-time quaking-induced conversion in the submandibular glands of Parkinson's disease patients. Mov Disord. 2020; 35:268-78.
15. Wang Z, Becker K, Donadio V, Siedlak S, Yuan J, Rezaee M, et al. Skin α-Synuclein Aggregation Seeding Activity as a Novel Biomarker for Parkinson Disease. JAMA Neurol [Internet]. 2021; 78:30. Available from: https://jamanetwork.com/journals/jamaneurology/fullarticle/2771032
16. van Rumund A, Green A J, Fairfoul G, Esselink R A, Bloem B R, Verbeek M M. Alpha-synuclein R T-QuIC in the CSF of uncertain cases of parkinsonism. Ann Neurol [Internet]. 2019; Available from: http://doi.wiley.com/10.1002/ana.25447
17. Rossi M, Candelise N, Baiardi S, Capellari S, Giannini G, Orrù C D, et al. Ultrasensitive R T-QuIC assay with high sensitivity and specificity for Lewy body-associated synucleinopathies. Acta Neuropathol [Internet]. Springer Berlin Heidelberg; 2020; 140:49-62. Available from: https://doi.org/10.1007/s00401-020-02160-8
18. Shahnawaz M, Mukherjee A, Pritzkow S, Mendez N, Rabadia P, Liu X, et al. Discriminating α-synuclein strains in Parkinson's disease and multiple system atrophy. Nature [Internet]. Nature; 2020 [cited 2020 Apr. 9]; 578:273-7. Available from: http://www.ncbi.nlm.nih.gov/pubmed/32025029
19. Metrick M A, do Carmo Ferreira N, Saijo E, Hughson A G, Kraus A, OrrG C, et al. Million-fold sensitivity enhancement in proteopathic seed amplification assays for biospecimens by Hofmeister ion comparisons. Proc Natl Acad Sci USA. 2019; 116:23029-39.
20. Kovacs G G. Molecular pathology of neurodegenerative diseases: Principles and practice. J Clin Pathol. 2019; 72:725-35.
21. Dujardin S, Commins C, Lathuiliere A, Beerepoot P, Fernandes A R, Kamath T V, et al. Tau molecular diversity contributes to clinical heterogeneity in Alzheimer's disease. Nat Med [Internet]. Springer U S; 2020; Available from: http://www.ncbi.nlm.nih.gov/pubmed/32572268
22. Klotz S, Fischer P, Hinterberger M, Ricken G, Hönigschnabl S, Gelpi E, et al. Multiple system aging-related tau astrogliopathy with complex proteinopathy in an oligosymptomatic octogenarian. Neuropathology. 2021; 41:72-83.
23. Kovacs G G, Wagner U, Dumont B, Pikkarainen M, Osman A A, Streichenberger N, et al. An antibody with high reactivity for disease-associated α-synuclein reveals extensive brain pathology. Acta Neuropathol. 2012; 124:37-50.
24. Lau A, So R W L, Lau H H C, Sang J C, Ruiz-Riquelme A, Fleck S C, et al. α-Synuclein strains target distinct brain regions and cell types. Nat Neurosci [Internet]. Springer U S; 2020; 23:21-31. Available from: http://dx.doi.org/10.1038/s41593-019-0541-x
25. Ozawa T, Paviour D, Quinn N P, Josephs K A, Sangha H, Kilford L, et al. The spectrum of pathological involvement of the striatonigral and olivopontocerebellar systems in multiple system atrophy: Clinicopathological correlations. Brain. 2004; 127:2657-71.
26. Inoue M, Yagishita S, Ryo M, Hasegawa K, Amano N, Matsushita M. The distribution and dynamic density of oligodendroglial cytoplasmic inclusions (GCIs) in multiple system atrophy: A correlation between the density of GCIs and the degree of involvement of striatonigral and olivopontocerebellar systems. Acta Neuropathol. 1997; 93:585-91.
27. Candelise N, Schmitz M, Llorens F, Villar-Piqué A, Cramm M, Thom T, et al. Seeding variability of different alpha synuclein strains in synucleinopathies. Ann Neurol. 2019; 85:691-703.
28. van Rumund A, Green A J E, Fairfoul G, Esselink R A J, Bloem B R, Verbeek M M. α-Synuclein real-time quaking-induced conversion in the cerebrospinal fluid of uncertain cases of parkinsonism. Ann Neurol. 2019; 85:777-81.
29. Ishizawa K, Ksiezak-Reding H, Davies P, Delacourte A, Tiseo P, Yen S H, et al. A double-labeling immunohistochemical study of tau exon 10 in Alzheimer's disease, progressive supranuclear palsy and Pick's disease. Acta Neuropathol. 2000; 100:235-44.
30. Singer W, Schmeichel A M, Shahnawaz M, Schmelzer J D, Boeve B F, Sletten D M, et al. Alpha-Synuclein Oligomers and Neurofilament Light Chain in Spinal Fluid Differentiate Multiple System Atrophy from Lewy Body Synucleinopathies. Ann Neurol. 2020; 88:503-12.
31. Candelise N, Schmitz M, Thüne K, Cramm M, Rabano A, Zafar S, et al. Effect of the micro-environment on α-synuclein conversion and implication in seeded conversion assays. Transl Neurodegener. Translational Neurodegeneration; 2020; 9:1-16.
32. Sekiya H, Kowa H, Koga H, Takata M, Satake W, Futamura N, et al. Wide distribution of alpha-synuclein oligomers in multiple system atrophy brain detected by proximity ligation. Acta Neuropathol [Internet]. Springer Berlin Heidelberg; 2019; 137:455-66. Available from: https://doi.org/10.1007/s00401-019-01961-w.
33. Dickson D W, Liu W K, Hardy J, Farrer M, Mehta N, Uitti R, et al. Widespread alterations of α-synuclein in multiple system atrophy. Am J Pathol [Internet]. American Society for Investigative Pathology; 1999; 155:1241-51. Available from: http://dx.doi.org/10.1016/S0002-9440(10)65226-1.
34. Campbell B C V, McLean C A, Culvenor J G, Gai W P, Blumbergs P C, Jäkälä P, et al. The solubility of α-synuclein in multiple system atrophy differs from that of dementia with Lewy bodies and Parkinson's disease. J Neurochem. 2001; 76:87-96.
35. Manca M, Kraus A. Defining the Protein Seeds of Neurodegeneration using Real-Time Quaking-Induced Conversion Assays. Biomolecules. 2020; 10.
36. Ferreira N, Gram H, Sorrentino Z A, Gregersen E, Ida S, Lasse S, et al. Multiple system atrophy—associated oligodendroglial protein p25α stimulates formation of novel α-synuclein strain with enhanced neurodegenerative potential. Acta Neuropathol [Internet]. Springer Berlin Heidelberg; 2021; Available from: https://doi.org/10.1007/s00401-021-02316-0.
37. Candelise N, Baiardi S, Franceschini A, Rossi M, Parchi P. Towards an improved early diagnosis of neurodegenerative diseases: The emerging role of in vitro conversion assays for protein amyloids. Acta Neuropathol Commun 2020; 8:1-16.
38. Manne S, Kondru N, Jin H, et al. Blinded R T-QuIC Analysis of α-SynucleinBiomarker in Skin Tissue From Parkinson's Disease Patients. Mov Disord 2020; 35:2230-9.
39. Mammana A, Baiardi S, Quadalti C, et al. R T-QuIC Detection of Pathological α-Synuclein in Skin Punches of Patients with Lewy Body Disease. Mov Disord 2021; 1-6.
40. Wang Z, Becker K, Donadio V, et al. Skin α-Synuclein Aggregation Seeding Activity as a Novel Biomarker for Parkinson Disease. JAMA Neurol 2021; 78:30.
41. Donadio V, Wang Z, Incensi A, et al. In Vivo Diagnosis of Synucleinopathies. Neurology 2021; 96: e2513-24.
42. Schmitz M, Canaslan S, Villar-Piqué A, et al. Validation of Plasma Neurofilament Light Chain as a Marker for α-Synucleinopathies. Mov Disord 2021; mds.28724.
43. Jecmenica Lukic M, Kurz C, Respondek G, et al. Copathology in Progressive Supranuclear Palsy: Does It Matter? Mov Disord 2020; 35:984-93.
44. Postuma R B, Berg D, Stern M, et al. MDS clinical diagnostic criteria for Parkinson's disease. Mov Disord 2015; 30:1591-601.
45. Gilman S, Wenning G K, Low P A, et al. Second consensus statement on the diagnosis of multiple system atrophy. Neurology 2008; 71:670-6.
46. Höglinger G U, Respondek G, Stamelou M, et al. Clinical diagnosis of progressive supranuclear palsy: The movement disorder society criteria. Mov Disord 2017; 32:853-64.
47. Byrne L M, Rodrigues F B, Blennow K, et al. Neurofilament light protein in blood as a potential biomarker of neurodegeneration in Huntington's disease: a retrospective cohort analysis. Lancet Neurol 2017; 16:601-9.
48. Doyle L, Wang M. Overview of Extracellular Vesicles, Their Origin, Composition, Purpose, and Methods for Exosome Isolation and Analysis. Cells. 2019; 8(7):727. doi:10.3390/cells8070727.
49. Younas N, Fernandez Flores L C, Hopfner F, Höglinger G U, Zerr I. A new paradigm for diagnosis of neurodegenerative diseases: peripheral exosomes of brain origin. Transl Neurodegener. 2022; 11(1):1-15. doi:10.1186/s40035-022-00301-5.

1. A method for differentiating movement disorders in a subject, comprising:
performing a seeding amplification assay for alpha-synuclein in a subject sample utilizing one of the following buffer conditions:
(i) NaCitrate, pH 7-9;
(ii) NaF, pH 7-9;
(iii) NaCLO₄, PH 3-5;
(iv) GndCl, pH 3-5; and
(v) MgCl₂, pH 3-5;
measuring the alpha-synuclein aggregation of the sample in the assay based on at least one of the following kinetic parameters: lag time, growth phase, T50, ThT max and AUC;
differentiating between Parkinson's Disease (PD) and Multiple System Atrophy (MSA) based on said sample kinetic parameters compared to control kinetic parameters, wherein control kinetic parameters for MSA show at least a two-fold difference from the control kinetic parameters for PD.

2. The method of claim 1, wherein the buffer condition is one of:
(i) Trisodium citrate, about pH8;
(ii) NaF, about pH8;
(iii) NaCLO₄, about pH4;
(iv) GndCl, about pH4; and
(v) MgCl₂, about pH4;

3. The method of claim 1, wherein the buffer condition is one of:
Trisodium citrate, about phi, PB;
(ii) NaF, about pH8, PB;
(iii) NaCLO₄, about pH4, Glycine;
(iv) GndCl, about pH4, Glycine; and
(v) MgC₁₂, about pH4, Glycine;

4. The method of claim 1, wherein the buffer condition is one of:
(i) Trisodium citrate, about pH8, about 40 mM PB;
(ii) NaF, about pH8, about 40 mM PB;
(iii) NaCLO₄, about pH4, about 50 mM Glycine;
(iv) GndCl, about pH4, about 50 mM Glycine; and
(v) MgCl₂, about pH4, about 50 mM Glycine;

5. The method of claim 1, wherein the buffer condition is (i).

6. The method of claim 5, wherein the concentration of Trisodium citrate is 50 nM to 500 nM or about 350 nm.

7. The method of claim 1, wherein the buffer condition is (ii).

8. The method of claim 8, wherein the concentration of NaCLO₄ is about 200 nm to 300 nm or about 250 nM.

9. The method of claim 1, further comprising the step of digesting the seeding amplification assay end products with thermolysin and wherein a MSA sample shows a 15 kDa product post-digestion and a PD sample does not show a distinct 15 kDa product post-digestion.

10. The method of claim 1, further comprising the step of digesting the seeding amplification assay end products with GndCl and comparing to control end product stability; wherein a MSA sample exhibits less stability to GndCl digestion than a PD sample.

11. The method of claim 1, wherein the sample from the subject has demonstrated alpha-synuclein seeding activity and the method further comprises:
measuring serum neurofilament light chain (NfL) concentration in the subject; and
further differentiating between PD and MSA based on said NfL concentration compared to control NfL concentrations, wherein control NfL concentrations for MSA are significantly increased compared to control NfL concentrations for PD.

12. The method of claim 1, for additionally differentiating progressive supranuclear palsy (PSP) from MSA and PD, the method further comprising:
measuring serum neurofilament light chain (NfL) concentration in the subject; and
differentiating progressive supranuclear palsy (PSP) from MSA and PD, wherein the sample from the subject demonstrated no/low alpha-synuclein seeding activity and said wherein a NfL measured concentration is compared to control NfL concentrations, wherein control NfL concentrations for PSP are significantly increased compared to control NfL concentrations for PD.

13. The method of claim 1, wherein the seeding amplification assay is Real-Time Quaking-Induced Conversion or Protein Misfolding Cyclic Amplification.

14. The method of claim 1, wherein the subject sample is brain, cerebrospinal fluid, skin, blood, saliva, urine, sebum, nasal secretions or nasal mucosal membrane, and extracellular vesicles (EVs) isolated from any of the foregoing.

15. The method of claim 1, wherein the subject sample is brain.

16. The method of claim 18, wherein the brain tissue is hippocampus-derived or amygdala-derived.

17. The method of claim 1, wherein the subject sample is skin.

18. The method of claim 1, wherein if the subject has been determined to have Parkinson's Disease, the method further comprises treating the subject for Parkinson's Disease.

19. The method of claim 1, wherein if the subject has been determined to have MSA, the method further comprises treating the subject for MSA.

20. The method of claim 1, wherein if the subject has been determined to have PSP, the method further comprises treating the subject for PSP.

21. A kit for performing a seeding amplification assay for alpha-synuclein, wherein the buffer included in said kit is as defined in claim 1.

22. The kit of claim 24, further comprising reagents to measure serum neurofilament light chain (NfL) concentration in a subject.
